SEQUENCE LISTING (6 sequences)

SEQ ID NO: 1 - DNA, 2130 bp, Homo sapiens
gctccggcggcttcgctgctagctcgcggcgacgtcgggccgattttcccaggatgacagagctgaggcagagggtggcccatgagccggttgcgccacccgaggacaaggagtcagagtcagaagcaaaggtagatggagagactgcatcggacagtgagagccgggcagaatccgcacccctgccagtctctgcagatgataccccggaggtcctcaatagggccctttccaacttgtcttcaagatggaagaactggtgggtgagaggcatcctgactttggccatgattgcatttttcttcatcatcatttacctgggaccaatggttttgatgataatcgtgatgtgcgttcagattaagtgtttccatgagataatcactattggctacaacgtctaccactcatatgatctgccctggttcaggacgctcagctggtactttctcctgtgtgtaaactatttcttctatggtgagacagtgacggattacttcttcaccctggtccagagagaagagcctttgcggattctcagtaaataccaccggttcatttcctttactctctatctaataggattctgcatgtttgtactgagtctggtcaagaagcattatcgactgcagttctacatgtttggctggacccatgtgacattgctgattgttgtaacacagtcacatcttgttatccacaacctatttgaaggaatgatctggttcattgtccccatatcttgtgtgatctgtaatgacatcatggcctatatgtttggctttttctttggtcggaccccactcatcaagctgtccccgaagaagacctgggaaggcttcattgggggcttctttgctactgtggtgtttggccttctgctgtcctatgtgatgtccgggtacagatgctttgtctgccctgtggagtacaacaatgacaccaacagcttcactgtggactgtgagccctcggacctgtttcgcctgcaggagtacaacattcctggggtgatccagtcagtcattggctggaaaacggtccggatgtaccccttccagattcacagcatcgctctctccacctttgcctcgctcattggcccctttggaggattcttcgcaagtggattcaaacgagcctttaaaatcaaagactttgccaataccattcctggccatggaggcatcatggatcgctttgactgccagtatctgatggccacctttgtcaatgtatacatcgccagttttatcagaggccctaacccaagcaaactgattcagcagttcctgactttacggccagatcagcagctccacatcttcaacacgctgcggtctcatctgatcgacaaagggatgctgacatccaccacagaggacgagtaggggccacccagggccaggagaacaggaacagaactgagcaggggcaggtctccaagaaatccctgcttggagctgcagaagggttgccttctgtaggtcggaggaatggaggcttactaaccaggtaagccttctatgcatccacaccaaaatcctgcagaatgtaagtaagctctgctttataagatgggttcaccttcatcgcagactgaaagtttcagtttttatttttttcagaaagcacgaaaaattatttataatagtctggagaaaaaacacactgtaatatttcaagtgtatgcagtagaatgtactgtaactgagccctttcccacatgtctaggctccaatgtctcctgtaggtccacctaactgtgtgttttcagggacaatgccatccatgtttgtgctgtagacttgctgctgctgaatcctttctggggactttctcatcgggcagggagcagagggcttctcgttcatgcaccctttgcctgaacacccatgtagctgctgtgttgtgtatatattactcttaagaggagtgtgtgtgtctgtgtttgttttaaaagtcacttatttcttacagtgatttcaattgcaccatgacttcttcactaaaaccacaaagtcctgcttaaaactatggaaaacctaacctgattagagccttgactattttgaagattaaatgcacactttttatataaaaaaaaaaaaaaaaaaaaaaaaaaaaaaa

SEQ ID NO: 2 - DNA, 2177 bp, Homo sapiens
ctgcggcgggacccgtgttctggaggcgactgctgggcctcctgcctggccgcccagggctggccgcgctcctgggacgcctgtccgaccgcctcggcaggaaccgggaccgccagcgcaggaggtcaccatggctgttattggctcccttgctgtccccagctgttccccaggtcacctccccaccttgctgcctgtgtccagaaggcgtgcaccggttccagtggatcagaaacctggttccagaatttggagtctccagttctcacgttagggtgctttcttccccggcagagtttttcgagctcatgaaggggcagataagagtagccaagaggcgggtcgtgatggcatccctctacctggggacaggtcctttggaacaggagctggtggactgcctggaaagtactctagaaaagtcactccaagcaaagtttccttcaaatctcaaggtctccattctcttagacttcacgcggggctcacgaggtcggaagaactcccgcacaatgctgctcccactcctgcggaggttcccagagcaggtccgagtctccctctttcacacgccgcacctccgtgggctgcttcggctcctcatccctgagcgcttcaacgagaccatcggcctccagcacattaaggtgtacctcttcgacaacagcgtcatcttgagcggtgcaaacctgagtgactcctacttcaccaaccgccaggaccgctacgtgttcctgcaggactgtgcggagattgccgacttcttcacggagctggtggacgcggtgggggatgtgtccctgcagctgcagggggacgacacggtgcaggtggtggatgggatggtgcatccttacaaaggggaccgggccgagtactgcaaggcagccaataagagggtcatggatgtgatcaactcagccaggacccgccagcagatgctgcatgcccagaccttccacagcaactctcttttgacccaggaagatgcagcagctgctggggatcgcagaccagcccctgacacctggatttatccgctgattcagatgaagcccttcgagattcaaatcgatgagattgtcactgagaccctgttgactgaggcggagcgcggggcaaaggtctacctcaccactggctatttcaacctgacccaggcctacatggacctggtcttgggcactcgggctgagtaccagatcctgctggcctcaccagaggtgaatggcttctttggggccaagggggtggccggcgccatcccagcggcctatgtgcacatcgagcgacagttcttcagtgaggtgtgcagcctgggacagcaggagcgggtccagcttcaggagtactggcggaggggctggacgttccacgccaaaggcctctggctgtacctggcagggagcagcctgccctgtctcacgctgattggctctcctaattttgggtacaggtcagttcaccgggacctggaggcccagattgcgatcgtgacggagaaccaggccctgcagcagcagcttcaccaggagcaagagcagctctacctgaggtcaggtgtggtgtcctctgccaccttcgagcagccgagtcgccaggtgaagctgtgggtgaagatggtgactccactgatcaagaacttcttctgaggacagacaggaatggccttgatgaagatgacaggcatggccggggtcagctctttcagccgcgcttcagcgatgactccagtctgggtgtcccagcgagcccctgcagggacagtatggctgagggtcaggtgtgctgccagtaagtgagggaggggctggcaggaagggtggggtcctcacactccccgccctctgcagagctgggctctaccccaaaaggcttcaggccagctgccacagctggaagcagaggccttcgtaggtgatggcctgcatgttgtaactaccccgtcccgctgggctcaaggaacagctcagctaaagccctcgggttccatccgtttaaatctgtggcattttcagagcctcatctgtcagccttaatgtcagtggcaggaagtcataactccagctaaaaattacagagtaaagttccctgattcttaatgtgtaatgtctgccctatgtgtacatacacaatataattatacatctgtgcatataaaaaaaaaaaaaaaaaaa

SEQ ID NO: 3 - DNA, 791 bp, Mus musculus
agctacacagcttgtgccatggactctgaggtcagcaacggttccggcctgggggccaagcacatcacagacctgctggtgttcggctttctccaaagctctggctgtactcgccaagagctggaggtgctgggtcgggaactgcctgtgcaagcttactgggaggcagacctcgaagacgagctgcagacagacggcagccaggccagccgctccttcaaccaaggaagaatagagccagattctgaaagtcaggaagaaatcatccacaacattgccagacatctcgcccaaataggcgatgagatggaccacaacatccagcccacactggtgagacagctagccgcacagttcatgaatggcagcctgtcggaggaagacaaaaggaactgcctggccaaagcccttgatgaggtgaagacagccttccccagagacatggagaacgacaaggccatgctgataatgacaatgctgttggccaaaaaagtggccagtcacgcaccatctttgctccgtgatgtcttccacacgactgtcaactttattaaccagaacctattctcctatgtgaggaacttggttagaaacgagatggactgaggagcccgcacaagcccgatggtgacactgcctccagaggaaccgcgaccatggaaagaccttggcctgaagacaggtcccagagaacagctgtctccctatttccaggtggtgggaaccccaagctggtgattcactggacatctctgcgttcagcttgagtgtatctgaagagtttacgccgg

SEQ ID NO: 4 - DNA, 2575 bp, Homo sapiens
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 5 - DNA, 765 bp, Shigella flexneri serotype 2a str. 2457T
atgcgttcgattgcccgacgtaccgcagtgggagctgcactattgcttgtcatgccagtagccgtatggatttctggctggcgttggcaacctggagaacaaagttggctactaaaagcggctttttgggttactgaaactgtcacccagccctggggcgtcattacacatttgattttgttcggctggtttctctggtgtctgcgttttcgcattaaggctgcctttgtattatttgccattctggcggcggcaatccttgttggacaaggcgttaaatcctggatcaaagacaaagtacaggaaccacgaccttttgttatctggttggaaaaaacacatcatattccggttgatgagttctacactttaaagcgagcagaacgcggaaatctagtaaaagaacagttggctgaagagaaaaatattccacaatatttgcgttcacactggcagaaagagacggggtttgcctttccttccggtcacacgatgtttgctgccagttgggcactgctggccgttggtttgctgtggccgcgtcggcgaacgttaaccattgctatcttgttggtctgggcaacgggagtcatgggaagccgcctgctgctcgggatgcattggccacgcgatctggtggtagctacgttgatttcgtgggcgctggtggcggtggcaacgtggcttgcgcaacgaatttgtgggccattaacaccacctgcggaagaaaatcgcgaaattgcacaacgagaacaagaaagttaa

SEQ ID NO: 6 - DNA, 1440 bp, Oceanobacillus iheyensis HTE831
atgggaataacctctttgttattaggactaacatttgttttaaatattgctttagctatttcaattatttttcttgaacgtaaagatccaacgtcctcttgggcatgggtaatggtccttttatttatacccatacttggattctttctttatttaatatttggcaaaccaatcagtaatagaaagattttttcttgggataagaagagccgtttgggagtaaagacaacagttcaatctcaattgagactcttagaagaaaatcaatttgaatttaatcaaccagaccttatcgagcataaggatcttgtatatttacatttgaaaaatgatgaagcaatttatacacagaataatggtgttgatatttttacagatggtcaaacgaaatttgatgctttgctagaagatatagaaaaagcgaaaaaacatatacatattcagtactatattatgcgcagtgatggcttaggaaataggcttgcagacatgctaataaaaaaagtaaatgaaggtgtagaagttagagttttatatgatgatatgggatcaagatcgttaaaaaacagttatataaaacgtttaaaaagagcaggagtgatggtcgaggcattcttcccatctcgattcatagtcaatttcaagattaattatcgaaatcaccgtaagctagcgattattgatggatatattggatatttaggtggttttaatgtaggcgatgaatatttagggattaataaaaagtttggatattggagagacacccatcttcgtgtgattggagatgcggtacaaagtatgcaaacacgttttatcctagattggaaccaagcatcgagggatactattttatataatgaagattattatcaaacagtatctgctggaaatgtcggtatgcaaattgttactagtggtcctgattcagaatatgaacaaatcaagaatggttatataaagatgatcatggaggcaaacgattatatttgtatccaaactccttattttattccggatgaaagtttaagggatgcattaaaaattgcagtattatctggggtacatgttaaaattatgataccgaacaaacctgatcatccatttgtatattgggcaacattatcgtattgtggcgatttaattcaagctggtgctgaaatatttatttatcagaatggatttttacatgctaaaacaatcattgtggatggtaggatagcttcagttggaacagctaatattgatgtacgtagtttccgacttaatttcgaagtaaatggcttcttgtatgattctgaagtagtgaatcggcttcaaaatgagttcgacgcagatttggagaaatctacgcaaatgacaaggaaactttatgatcaaagatcgataggaatccgttttaaagaatctatatccaggttgatttcacctgttttataa

(19) United States
(12) Patent Application Publication
(10) Pub. No.: US 2006/0078560 A1
(43) Pub. Date: Apr. 13, 2006

(54) Method of inducing apoptosis and inhibiting cardiolipin synthesis

(75) Inventors: Haris Jamil, Libertyville, IL (US); Moghis U. Ahmad, Wadsworth, IL (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NeoPharm, Inc., Lake Forest, IL (US)

(21) Appl. No.: 11/287,530

(22) Filed: Nov. 22, 2005

Related U.S. Application Data
(63) Continuation of application No. PCT/US04/20104, filed on Jun. 23, 2004.
(60) Provisional application No. 60/480,669, filed on Jun. 23, 2003.

(51) Int. Cl. A61K 39/395; A61K 31/685

(52) U.S. Cl. 424/146.1; 514/78

(57) ABSTRACT

The present invention provides a method for inducing apoptosis within a cell by exposing the cell to an inhibitor of cardiolipin synthesis under conditions sufficient to induce apoptosis within the cell. The method can be used to investigate or treat disorders such as cancer, obesity, and cardiovascular disorders. The invention also provides a pharmaceutical composition including an inhibitor of cardiolipin synthesis and a liposomal carrier.

### CROSS-REFERENCE TO RELATED APPLICATIONS

This patent application is a continuation of International Patent Application No. PCt/US2004/020104, filed Jun. 23, 2004, which claims the benefit of U.S. Provisional Patent Application No. 60/480,669, filed Jun. 23, 2003, the disclosures of which are incorporated herein by reference.

### FIELD OF THE INVENTION

This invention pertains to a method of inducing apoptosis, principally via inhibiting the synthesis of cardiolipin, and therapeutic uses thereof.

### BACKGROUND OF THE INVENTION

Apoptosis, or programmed cell death, is an evolutionarily conserved mechanism of cell death that has a crucial role in various biological events, including development, the maintenance of homeostasis and the removal of obsolete cells [Reed, “Bcl-2 family proteins,” Oncogene, 17 (25), 3225-3236 (1998); Kroemer et al., “The mitochondrial death/life regulator in apoptosis and necrosis,” Annu. Rev. Physiol., 60, 619-642 (1998); Skulachev, “Cytochrome c in the apoptotic and antioxidant cascades,” FEBS Lett., 423 (3), 275-280 (1998)]. Apoptotic signals are activated by various stimuli and converge towards a common death pathway, in which proteins in the Bcl-2 family act as regulators, and proteases in the caspase family act as signal transducers [Reed, supra; Kroemer et al., supra, Skulachev, supra]. Recent evidence has shown that mitochondria have a crucial role in apoptosis by releasing apoptotic factors such as cytochrome c and apoptosis-inducing factor from the intermembrane space into cytoplasm.

Apoptosis may occur by two general pathways, i.e. receptor-mediated and stress-induced (mitochondrial-initiated) apoptosis [Budihardjo et al., “Biochemical pathways of caspase activation during apoptosis,” Annu. Rev. Cell Dev. Biol., 15, 269-290 (1999)]. In both pathways, cytochrome c release is one of the most important regulatory steps. In receptor-mediated apoptosis, caspase 8 is activated early and cleaves BID [Luo et al., “BID, a Bcl2 interacting protein, mediates cytochrome c release from mitochondria in response to activation of cell surface death receptors,” Cell, 94 (4), 481-490 (1998)]. After cleavage by caspase 8, the carboxy-terminal portion (tBid) moves from cytosol to mitochondria, where it induces release of cytochrome c. BID also appears to modulate lipid transfer between ER and mitochondria [Degli Esposti et al., “Bid, a widely expressed proapoptotic protein of the Bcl-2 family, displays lipid transfer activity,” Mol. Cell. Biol., 21 (21), 7268-7276 (2001)].

In stress-induced apoptosis, however, caspase 8 is usually not activated, and the mechanism of cytochrome c release is uncertain. Current theories involve transient opening of the mitochondria permeability transition pore causing slight swelling as well as formation of pores in the outer membrane by proapoptotic members of the Bcl-2 family, e.g. BAX and BAK [Budihardjo et al., supra; Bernardi et al., “Mitochondria and cell death: Mechanistic aspects and methodological issues,” Eur. J. Biochem., 264 (3), 687-701 (1999); Wei et al., “Proapoptotic BAX and BAK: a requisite gateway to mitochondrial dysfunction and death,” Science, 292 (5517), 727-730 (2001)]. These mechanisms mediate the passage of unbound cytochrome c through the mitochondrial outer membrane. Cytochrome c is bound to the outer surface of the inner membrane phospholipids by electrostatic forces (predominating at neutral pH). Dissociation from the inner membrane is a necessary first step before cytochrome c can pass through release of channels and ultimately reach the cytosol. The released cytochrome c activates caspase 9 in concert with the cytosolic factors ATP and Afaf-1, and, as a result, caspase 3 is activated [Li et al., “Cytochrome c and dATP-dependent formation of Apaf-1/caspase-9 complex initiates an apoptotic protease cascade,” Cell, 91 (4), 479-489 (1997)]. Apoptosis-inducing factor has also been reported to induce apoptotic changes in the nucleus [Susin et al., “Molecular characterization of mitochondrial apoptosis-inducing factor,” Nature, 397 (6718), 441-446 (1999)]. The anti-apoptotic proteins Bcl-2 and Bcl-xL, which are localized predominantly in mitochondrial outer membranes, inhibit the release of cytochrome c from mitochondria [Reed, supra]. Although cytochrome c normally shuttles electrons between complex III (cytochrome c reductase) and complex IV (cytochrome c oxidase) of the respiratory chain, cytochrome c released from mitochondria is an important proapoptotic signal [Kroemer et al., supra; Skulachev, supra] in the mitochondrial death pathway.

The failure of cells to undergo programmed cell death is implicated in tumorigenesis in a variety of human malignancies. Cells that have accumulated high levels of DNA damage are eliminated from the organism via programmed cell death without negatively affecting the surrounding tissue. Disruption of programmed cell death in a cell greatly increases the chance of that cell becoming tumorgenic, since the damage can cause mutations that lead to malignant transformation. In addition, programmed cell death appears to be a first line of defense against the proliferation of cells that might form a tumor: cells in which growth control is dysregulated in a way that could result in uncontrolled proliferation are generally able to recognize that aberrant state and commit suicide by programmed cell death. If programmed cell death is blocked in such cells, cancer could arise. The failure to undergo programmed cell death per se can even lead to excessive number of cells and cancer: e.g., as the result of inappropriate activation of the Bcl-2 gene, a suppressor of programmed cell death, most follicular B cell lymphomas result in the accumulation of excessive number of cells that would normally undergo programmed cell death. Many tumor cell types also appear to require Bcl-2 expression to avoid apoptosis and remain proliferative. Thus, the inability to regulate programmed cell death may be a key causative even in many, and perhaps all, cancers. Regulation of apoptosis also may be important for other disorders, such as obesity and cardiovascular disorders characterized by fatty plaque buildup in the walls of vessels. Thus, there is a need for methods for regulating apoptosis.

### BRIEF SUMMARY OF THE INVENTION

The present invention provides a method for inducing apoptosis within a cell by exposing the cell to an inhibitor of cardiolipin synthesis under conditions sufficient to induce apoptosis within the cell. The method can be used to investigate or treat disorders such as cancer, obesity, and cardiovascular disorders. The invention also provides a pharmaceutical composition including an inhibitor of cardiolipin synthesis and a liposomal carrier. These and other advantages of the invention, as well as additional inventive features, will be apparent upon reading the following detailed description of the invention.

### DESCRIPTION OF THE FIGURES

FIG. 1 depicts the structure of cardiolipin

FIG. 2 is a flowchart depicting the cardiolipin synthetic pathway.

### DETAILED DESCRIPTION OF THE INVENTION

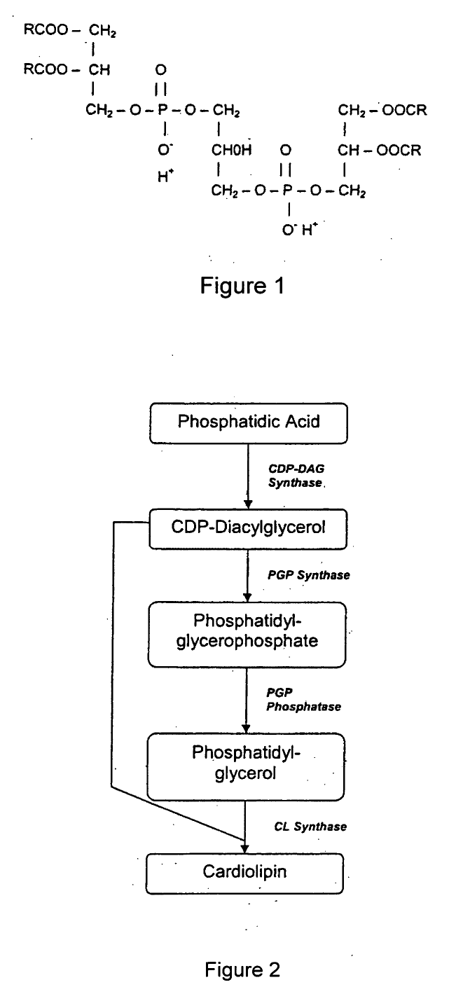

The invention provides a method of inducing apoptosis in a cell. In accordance with the inventive method, the cell is exposed to an inhibitor of cardiolipin synthesis under conditions sufficient to induce apoptosis within the cell. Cardiolipin (FIG. 1) is a unique dimeric phospholipid that contains four acyl groups and two negative charges. The name “cardiolipin” is derived from the fact that the compound was first found in animal hearts, where it is especially abundant. However, cardiolipin is found almost exclusively in mitochondria and bacteria, i.e. those whose function is to generate an electrochemical potential for substrate transport and ATP synthesis, and can account for as much as 20% of mitochondrial lipids [Pangbom, “Isolation and purification of a serologically active phospholipid from beef heart,” J. Biol. Chem., 143, 247-256 (1942)]. Cardiolipin accounts for about 10% of the phospholipids of bovine heart muscle. In animal tissues, cardiolipin contains almost exclusively 18 carbon fatty acids, and 80% of this is typically linoleic acid.

Cardiolipin is a specific lipid component of mitochondria and its biological function in this organelle is clearly crucial. Cardiolipin is located mainly on the inner membrane of mitochondria, where it interacts with a large number of mitochondrial proteins, such as NADH: ubiquinone oxidoreductase, cytochrome. c oxidase and cytochrome c. This interaction effects functional activation of certain enzymes, especially those involved in oxidative phosphorylation. Indeed, many of the mitochondrial protein complexes contain cardiolipin molecules integrated into their quaternary structure, where they are essential components of the interface between the complex and its environment or between subunits within the complex. Removal of cardiolipin leads to the break-up of the complex and loss of functionality.

Cardiolipin is the most unsaturated lipid in the human body due to a process of constant re-modeling integrated between mitochondria and ER [Schlame et al., “Lysocardiolipin formation and reacylation in isolated rat liver mitochondria,” Biochem. J., 272, 589-595 (1990); Kajiyama et al., “Protection by verapamil of mitochondrial glutathione equilibrium and phospholipid changes during reperfusion of ischemic canine myocardium,” Circ. Res., 61 (2), 301-310 (1987)]. Cardiolipin is normally re-modeled by its de-acylation to mono and di-lysocardiolipin that need to be transported to the ER for efficient reacylation by acyltransferases. Cardiolipin biosynthesis (FIG. 2) is restricted to the inner mitochondria membrane [Hatch, “Cardiolipin: biosynthesis, remodeling and trafficking in the heart and mammalian cells,” Int. J. Mol. Med., 1 (1), 33-41 (1998)]. After the conversion of phosphatidic acid (PA) plus CTP to CDP-diacylglycerol (DAG) and pyrophosphate by CDP-DAG synthase, cardiolipin biosynthesis in eukaryotes occurs in a three-step process. First, phosphatidylglycerophosphate (PGP) synthase catalyzes the formation of PGP from CDP-DAG and 5-glycerol-3-phosphate. In the second step, PGP is dephosphorylated to phosphatidylglycerol (PG) by PGP phosphatase. Lastly, cardiolipin synthase catalyzes a phosphatidyl transfer from CDP-DAG to PG, an irreversible reaction that involves cleavage of a high energy anhydride bond to form cardiolipin. Interestingly, BID preferentially interacts with negatively-charged phospholipid phosphatidylglycerol, a precursor of cardiolipin synthesis [Hatch, supra]. This suggests that BID also may be involved in synthesis, or recycling, of cardiolipin.

The regulation of cardiolipin synthesis is important for mitochondrial function in the life cycle of the mammalian cell. Cytochrome c (a proapoptic factor that binds preferentially to cardiolipin but not to cardiolipin hydroperoxide) associates strongly with cardiolipin [Demel et al., “Differential interactions of apo- and holocytochrome c with acidic membrane lipids in model systems and the implications for their import into mitochondria,” J. Biol. Chem., 264 (7), 3988-3997 (1989).]. Ostrander et al. [Ostrander et al., “Decreased cardiolipin synthesis corresponds with cytochrome c release in palmitate-induced cardiomyocyte apoptosis,” J. Biol. Chem., 276 (41), 38061-38067 (2001)] demonstrated that cardiolipin synthesis is directly correlated with release of cytochrome c. Reactive oxygen species (ROS) generated during mitochondrial respiration might be expected to induce the peroxidation of cardiolipin because cardiolipin in mitochondria contains significant quantities of highly unsaturated fatty acids. In a recent study it was observed that peroxidation of cardiolipin in the mitochondria resulted in the dissociation of cytochrome c from mitochondrial inner membranes, the initial step in the release of cytochrome c from mitochondria [Vik et al., “Diphosphatidylglycerol is required for optimal activity of beef heart cytochrome c oxidase,” Proc. Natl. Acad. Sci. USA, 78 (3), 1456-1460 (1981); Ostrander et al., supra; Sprong et al. “How proteins move lipids and lipids move proteins,” Nat. Rev. Mol. Cell. Biol., 2 (7), 504-513 (2001)]. The quantitative interaction of cytochrome c with anionic mitochondrial phospholipids may predetermine the relative distribution of this protein between mitochondrial membranes and the cytochrome c in energy production and programmed cell death. Evidence has accumulated that cardiolipin plays an integral role as an upstream effectors of these important processes [Sparagna et al., “A metabolic role for mitochondria in palmitate-induced cardiac myocyte apoptosis,” Am. J. Physiol. Heart Circ. Physiol., 279, H2124-H2132 (2000); Watts et al., “On the complexities of ceramide changes in cells undergoing apoptosis: lack of evidence for a second messenger function in apoptotic induction,” Cell Death Differ., 6 (2), 105-114 (1999); Listenberger et al., “Palmitate-induced apoptosis can occur through a ceramide-independent pathway,” J. Biol. Chem., 276 (18), 14890-14895 (2001)].

Without being bound by any particular theory, it is believed that inhibition of cardiolipin synthesis in accordance with the inventive method may induce apoptosis by interfering with the ability of BID to target mitochondria [see Lutter et al., “Cardiolipin provides specificity for targeting of tBid to mitochondria,” Nat. Cell Biol., 2 (10), 754-756 (2000); Listenberger et al., supra], thus impairing the lipid transfer between the ER and mitochondria. Moreover, the biosynthesis of cardiolipin has been found to be critically affected in a model of lipid-induced apoptosis [Kajiyama et al., supra], consistent with the decrease of mitochondrial cardiolipin content during apoptosis induced by various stimuli [Nomura et al., “Mitochondrial phospholipid hydroperoxide glutathione peroxidase inhibits the release of cytochrome c from mitochondria by suppressing the peroxidation of cardiolipin in hypoglycaemia-induced apoptosis,” Biochem. J., 351, 183-193 (2000)]. Indeed, several recent studies have probed a link between cardiolipin levels and apoptosis. For example, in staurosporine-treated granulose cells undergoing apoptosis, cardiolipin levels were observed to be reduced [Khan et al., “Mitochondria and caspases in induced apoptosis in human luteinized granulosa cells,” Biochem. Biophys. Res. Comm., 269 (2), 542-545 (2000)]. Peroxidation of cardiolipin induced release of cytochrome c from mitochondria into the cytosol and this was associated with the induction of apoptosis [Shidoji et al., “Loss of molecular interaction between cytochrome c and cardiolipin due to lipid peroxidation,” Biochem. Biophys. Res. Comm., 264 (2), 343-347 (1999); Ushmorov et al., “Nitric-oxide-induced apoptosis in human leukemic lines requires mitochondrial lipid degradation and cytochrome C release,” Blood, 93 (7), 2342-2352 (1999); Poot et al., “Analysis of Mitochondria by Flow Cytometry,” in Cytometry (Third edition, Part B, Vol. 64) (Darzynkiewicz et al., eds.), Chapter 35, 311-317 (Academic Press, San Diego, Calif., 2000)]. Suppression of cardiolipin peroxidation also inhibits release of cytochrome c from mitochondria [Nomura et al., supra].

In accordance with the inventive method, the cell is exposed to an inhibitor of cardiolipin synthesis. Any agent able to inhibit the production of cardiolipin can be employed in the context of the present invention. For example, several compounds that impact cardiolipin synthesis are known in the art, many of which can be suitably used in the context of the inventive method. One exemplary compound is 1-Decanoyl-sn-glycero-3-phosphorylcholine [Schlame et al., “Cardiolipin is synthesized on the matrix side of the inner membrane in rat liver mitochondria,” J. Biol. Chem., 268 (1), 74-79 (1993)]. Another compound for use in the inventive method is 1-O-octadecyl-2-O-methyl-rac-glycero-3-phosphocholine [Cabener et al., “Induction of apoptosis in human mitogen-activated peripheral blood T-lymphocytes by the ether phospholipid ET-18-OCH3: involvement of the Fas receptor/ligand system,” Br. J. Pharmacol., 127 (4), 813-825 (1999)]. Another compound for use in the inventive method is Hexadecylphosphocholine [Wieder et al., “Induction of ceramide-mediated apoptosis by the anticancer phospholipid analog, hexadecylphosphocholine,” J. Biol. Chem., 273 (18), 11025-11031 (1998)]. Yet another compound that can be used in the inventive method is Lysophosphatidic acid, [Gueguen et al., “A lysophosphatidic acid analogue is revealed as a potent inhibitor of phosphatidylcholine synthesis, inducing apoptosis,” Biochem. J., 368, 447-459 (2002)]. Palmitate is known to diminish the content of mitochondrial synthesis of cardiolipin [Ostrander et al., supra] and it can be used as the inhibitor of cardiolipin in the context of the inventive method. Yet another suitable compound for use in the inventive method is N-(4-hydroxyphenyl)retinamide, which induces oxidation of cardiolipin and leakage of mitochondria and can cause gradual decrease in mitochondrial oxidative turnover and cardiolipin level [Poot et al., “Distinct patterns of mitochondrial changes precede induction of apoptosis by all-trans-retinoic acid and N-(4-hydroxyphenyl)retinamide in MCF7 breast cancer cells,” Exp. Cell Res., 279 (1), 128-140 (2002)]. Phosphatidyl-3,4-dihydroxybutyl-1-phosphate, which is an analog of glycerol-3-phosphate [Lacombe et al., “Effect of 3,4-dihydroxybutyl-1-phosphonate on cardiolipin synthesis in B. subtilis,” Biochim. Biophys. Acta, 1005 (2), 103-108 (1989)], also can be used as the inhibitor of cardiolipin synthesis in the context of the inventive method. Yet another compound that can be used in the context of the inventive method is phosphatidylserine [Uchida et al., “Induction of apoptosis by phosphatidylserine,” J. Biochem. (Tokyo), 123 (6), 1073-1078 (1998)]. Sphingosine-1-phosphate [Grey et al., “The phospholipids sphingosine-1-phosphate and lysophosphatidic acid prevent apoptosis in osteoblastic cells via a signaling pathway involving G(i) proteins and phosphatidylinositol-3 kinase,” Endocrinology, 143 (12), 4755-4763 (2002)] also can be used as the inhibitor in the context of the inventive method). Another compound that can be used as the inhibitor of cardiolipin synthesis is sulfoquinovosyldiacylglycerol [Quasney et al., “Inhibition of proliferation and induction of apoptosis in SNU-1 human gastric cancer cells by the plant sulfolipid, sulfoquinovosyldiacylglycerol,” J. Nutr. Biochem., 12 (5), 310-315 (2001)]. Preferred compounds include 1-Decanoyl-,y-glycero-3-phosphorylcholine, Lysophosphatidic acid, and Phosphatidyl-3,4-dihydroxy butyl-1-phosphate.

For use in vivo, the dosage of any of the foregoing compounds will depend on its manner of formulation and administration. However, optimizing the dosage of such compounds to suit a particular application of the inventive method is within the ordinary skill of the art. Generally, however, the dosage of such compounds for intravenous administration can be as little as about 0.1 μg/kg, such as little as about 0.5 μg/kg, or as little as about 1 μg/kg, and more typically as little as about 2 μg/kg or as little as about 5 μg/kg or even as little as about 10 μg/kg, such as as little as about 25 μg/kg or about 50 μg/kg or about 100 μg/kg. Some compounds can be administered in dosages as little as about 250 μg/kg or as little as about 500 μg/kg. It may be desirable to administer one or more of the compounds in dosages as little as about 1 mg/kg, such as little as about 2 mg/kg or as little as about 5 mg/kg or 10 mg/kg. Some compounds can be administered in dosages of as little as about 10 mg/kg, such as, as little as about 25 mg/kg or about 50 mg/kg or about 100 mg/kg. Some compounds can be administered in dosages as little as about 250 mg/kg or as little as about 500 mg/kg. The maximum tolerated dose of such compounds can be ascertained by those of skill in the art. Moreover, for intravenous injection, the dosage can be administered continuously for several days or several hours (e.g., about 12 hours or about one or a few hours). For bolus administration, such compounds may be effectively provided to a patient in bolus injections of a few minutes or less, preferably less than one or two minutes, and even more preferably less than about 30 seconds, such as less than about 20 seconds or even less than about 10 or about 5 seconds. Dosages for bolus injections can range from as little as 0.1 μg or μg less, or as little as about 0.5 μg-1 μg, such as as little as about 2 μg or as little as about 5 μg or 10 μg, or even as little as about 25 μg or 50 μg or 100 μg. Some compounds can be administered as a bolus in dosages as little as about 250 μg or as little as about 500 μg. It may be desirable to administer one or more of the compounds as a bolus in dosages as little as about 1 mg, such as little as about 2 mg or as little as about 5 mg or 10 mg. Some compounds can be administered via bolus injection in dosages of as little as about 10 mg, such as, as little as about 25 mg or about 50 mg or about 100 mg. For bolus injection some compounds can be administered in dosages as little as about 250 mg or as little as about 500 mg. Higher dosages are possible, in some applications, and the optimal dosage can be determined by a skilled artisan without the use of undue experimentation.

In another embodiment, cardiolipin synthesis can be inhibited via recombinant DNA technology, such as via antisense inactivation of the production of one or more enzymes in the cardiolipin synthesis pathway (see FIG. 2). Antisense inhibition can be achieved using a polynucleotide (e.g., an oligonucleotide) having a sequence consisting essentially of at least a portion of a gene encoding an enzyme involved in the synthesis of cardiolipin. Of course, more than one antisense polynucleotide can be used in concert to achieve redundant expression of the same gene, or to target more than one of the genes involved in the cardiolipin synthetic pathway.

The portion of the desired gene to which the polynucleotide is antisense can be a coding sequence or a regulatory sequence, such as a 5′ or 3′ untranslated region, an intron, an exon, a region including a start site or a transcription or translation termination site. Moreover, while the antisense polynucleotide need not be an exact complement of the region of the gene, it should be able to bind to the gene RNA sequence within the cell to attenuate or inhibit expression of the gene encoding the inhibitor of cardiolipin synthesis. Thus, while an exact complement is not required, typically the antisense polynucleotide is an exact complement of at least a portion of a gene encoding an enzyme involved in the synthesis of cardiolipin. While the design of antisense polynucleotides is within the ordinary skill in the art, generally, the antisense polynucleotide will contain at least about 8, and more preferably at least about 12 nucleotides, such as at least about 15 or at least about 20 nucleotides. Antisense polynucleotides containing as many as about 25 or as many as about 30 nucleotides also can be employed, and, indeed, the antisense polynucleotide can contain a larger number of nucleotides, if desired, such as about 40 or about 50 or more nucleotide bases. Generally, however, the antisense polynucleotide contains between about 10 and about 50 nucleotides (such as between about 15 and about 40 nucleotides), while longer or shorter polynucleotides (even substantially longer or shorter) can be employed.

It is within the ordinary skill of the art to design and produce polynucleotides to achieve antisense inhibition of target genes. Moreover, the genetic sequences of the enzymes catalyzing the synthesis of cardiolipin (e.g., phosphatidylglycerophosphate synthase, phosphatidylglycerophosphate phosphatase, phosphatidate cytidylyltransferase 2, cardiolipin synthase, and BID) are known (see, for example, NCBI Entrez Accession No. BC025751 (SEQ ID NO:1), NM_{—}024419 (SEQ ID NO:2), U75506 (SEQ ID NO:3), NMJ38578 (SEQ ID NO:4), NC_{—}004741 (SEQ ID NO:5), and AP004603(SEQ ID NO:6), which are the sequences published by NCBI as of Jun. 23, 2003. Thus, exemplary antisense polynucleotides have sequences consisting of 12-40 base pairs complementary to any of these published sequences. Using these known sequences of these published sequences, an antisense polynucleotide can be derived and constructed using any desired methodology, such as rtPCR of a cDNA library using primers flanking the desired sequence, or using automated oligodeoxyribnucleotide synthesis machines.

To achieve antisense inhibition, the antisense polynucleotide is introduced into the desired cells such that it is able to interact with the desired RNA target within the cells (e.g., the gene encoding the enzyme involved in the synthesis of cardiolipin). Thus, the antisense sequence can be introduced into the cells directly as “naked” DNA polynucleotides that can be taken up into the cells. Alternatively, the antisense sequence can be engineered into a genetic vector, such as a plasmid or viral vector (e.g., adenoviral, vaccinea viral, herpesviral, retroviral or other suitable viral vector), for efficient transfection or infection of the target cells. In this respect, the antisense polynucleotide can be produced within the cells by engineering the desired antisense sequence into an expression vector operably linked to a promoter for expression within the cells. Once introduced into the cells, the antisense oligonucleootide attenuates or inhibits the production of cardiolipin within the cells, thus promoting apoptosis.

The inventive method can be employed on a cell, tissue, or organ explant in vitro, or in vivo. The cell type can be of any desired type, such as tissue culture cells, cancer cells, adipose cells, and vascular smooth muscle and endothelial cells.

When used in vitro, the method can serve as an investigative tool to study apoptosis and the regulation thereof in tissue culture cells, organ explants, etc. In this regard, for in vitro application, cultured cells, explanted tissues containing cells, artificial tissues or organ components, or even explanted organs can be bathed in tissue culture media containing one or more inhibitors of cardiolipin synthesis under conditions permitting the inhibitor of cardiolipin synthesis to contact the cells in question, for example, as discussed above. Vascular tissues, or structures (e.g., organ explants, tissue explants, or artificial organs or tissues) containing vascular vessels or other cavities can alternatively be perfused with a suitable medium containing an inhibitor of cardiolipin synthesis. Thus employed, the method can be used to probe the dosing, time course, and other parameters affecting the inhibition of cardiolipin synthesis within cells. Alternatively, cells, tissues, organs or other structures treated in accordance with the inventive method in vitro can, in some applications, be implanted into a host for therapeutic applications.

The inventive method also can be used in vivo for therapeutic treatment of disorders within human or animal patients. In one embodiment, the inventive method can be employed in vivo against adipose tissue, and the “cell” or cells to be treated in accordance with the inventive method can be adipose cell(s). Alternatively, the cell within the adipose tissue can be a connective tissue cell, cells in the stromal-vascular portion of the adipose tissue, or other cells within the adipose tissue. Where employed against adipose tissue, the inventive method can be used to attenuate the progression of obesity in a patient suffering from obesity. In this regard, the inhibitor of cardiolipin synthesis can cause apoptosis within the adipose tissue (e.g., of adipose cells, connective tissue, and/or stromal or other cells) and/or inhibit the proliferation or growth of such cells within the patient, which can reduce the volume of (or at least retard the growth of) adipose tissue within the patient. In this regard, the inventive method can attenuate the progression of obesity (e.g., adipose growths) within the patient. As with the treatment of cancers and tumors noted above, a preferred method for introducing the inhibitor of cardiolipin synthesis within adipose tissue is via direct inter-tissue (e.g., interstitial) injunction, such as via convection-enhanced delivery.

In attenuating the progression of obesity within the patient, the inventive method need not achieve reduction in obesity, although this is preferred. Indeed, it is often sufficient for the inventive method to reduce the progression of the disease within the patient. However, it is desirable for the inventive method to achieve remission of the disorder or even to reverse obesity in some patients, e.g., leading to a reduction in adipose tissue volume or mass, and a loss of weight for the patient. Moreover, it will be understood that the inventive method can be used in conjunction with, or adjunctively with, drugs or pharmaceutical agents that also treat obesity. Similarly, the inventive method can be used in conjunction with surgical procedures, dietary and behavior modification therapy, and other strategies for treating obesity within afflicted patients.

In another embodiment, the inventive method can be directed against cells of the cardiovascular system, for example, vascular smooth muscle cells and endothelial cells. Such cells typically are within the lumens of the vasculature (e.g., arterial lumens, venous lumens, etc.). Thus employed, the inventive method can be used to treat a patient suffering from a cardiovascular disease. Exemplary cardiovascular diseases that can be treated in accordance with the inventive method include those characterized by the buildup of fatty plaque deposits in vascular walls. In accordance with the inventive method, the inhibitor of cardiolipin synthesis (e.g., within a suitable composition also including a pharmaceutically-acceptable carrier) is administered to the patient under conditions sufficient to inhibit proliferation of fatty plaque deposits in vascular walls. For example, by inducing apoptosis within such cells, the inventive method can retard the proliferation of these cells within the vascular tissue. While it is sufficient for the inventive method to attenuate the proliferation of such cells, in some embodiments, the inventive method can halt the proliferation of such cells, and thereby block the continued build-up of fatty plaque within the vessel lumen. It is more preferred for the inventive method to reduce the number of such cells, and thereby achieve a reduction in the amount of plaque present within the vascular tissue.

For treatment of cardiovascular diseases, typically the composition including the inhibitor of cardiolipin synthesis is delivered to the patient in situ within a desired site of a blood vessel. For in situ delivery of a vector internally, the region of interest desirably is further segregated from the remainder of the patient's tissue. Any of a variety of known surgical procedures for physically segregating the region of interest is appropriate. Various endovascular surgical techniques appropriate for segregating a region of interest are available, depending upon the location of the target. Endovascular surgical procedures include, but are not limited to, balloon angioplasty, intravascular stents, laser-assisted balloon angioplasty, double balloon catheterization, mechanical endarterectomy and vascular endoscopy. For a review of endovascular alternatives, see generally Ahn, “Endovascular Surgery,” in Vascular Surgery, A Comprehensive Review (4th ed.), (Moore et al., eds.) (W. B. Saunders & Co., Philadelphia, Pa., 1993).

Several catheter designs can be utilized for local delivery of a composition including an inhibitor of cardiolipin synthesis to the patient. One catheter design consists of two independently inflated balloons, one proximal and one distal to the vascular delivery site. Inflation of these balloons provides an evacuated isolated arterial segment into which a composition including an inhibitor of cardiolipin synthesis can be infused. This system is, however, limited by a failure to provide distal arterial perfusion. A second catheter design developed by Wolinsky allows the infusion of the composition including an inhibitor of cardiolipin synthesis through 25-100 μM pores under pressures up to 5 atm. This perfusion pressure increases the depth of penetration by the composition including an inhibitor of cardiolipin synthesis and, where the inhibitor is a genetic vector, can additionally increase the transfer efficiency of the vector into the cells. Yet another catheter design utilizes an expandable stent, which traps the balloon against the arterial wall and allows intramural delivery of the composition including an inhibitor of cardiolipin synthesis through spaces in the stent material. Additionally, these stents can be modified with burrs, which create holes deeper in the vessel wall and allow flow of the composition including an inhibitor of cardiolipin synthesis to these sites to allow more uniform delivery throughout the vessel wall. Also, biodegradable stents formed from agents such an ethylenevinyl acetic copolymer are appropriate for localized delivery to vascular tissue. Alternatively, an intravascular stent can be utilized wherein the endovascular scaffold of the stent is bathed in a ointment, cream, lotion, colloidal dispersion such as a gel or magma or any other acceptable carrier which comprises the inhibitor of cardiolipin synthesis for delivery to the targeted portion of a vessel segment. This solution is applicable to either an in situ or ex vivo based vessel delivery. Another specific application, offered for the purpose of example and not of limitation, is the use of a self-expanding stent. This intravascular stent can be bathed in a gel solution comprising an inhibitor of cardiolipin synthesis and delivered percutaneously to the target vessel site. An initial angioplasty, if necessary, is followed by delivery of the bathed scaffold to the target vessel site. The delivery catheter is removed and the scaffold is dilated with a conventional balloon. It is within the purview of the skilled vascular surgeon to use other types of intravascular stents such as a balloon expandable stent or a thermal expanding stent. Additionally, numerous balloon catheters of varying sizes, shapes, and types are available to the skilled vascular surgeon for endovascular delivery of the composition including an inhibitor of cardiolipin synthesis.

The inventive method, of course, can be employed in connection with surgical endovascular techniques, such as procedures to bypass a vascular occlusion. Such procedures typically involve a homograft or heterograft comprising an artery or vein, or a segment thereof, or an artificial conduit. Vascular bypass procedures involve forming a proximal and distal anastomosis between the graft conduit and the vessel. A composition including an inhibitor of cardiolipin synthesis then can be transferred to the cells in the region of the anastomoses to promote proper healing of the surgical wound between the two conduits. Where the graft conduit is not artificial (e.g., an artery, a vein, or a segment thereof), the composition including an inhibitor of cardiolipin synthesis can be transferred to the cells of the graft lumen. Additional preferred methods for delivering a composition including an inhibitor of cardiolipin synthesis to a vessel in vivo or ex vivo involve vascular surgery.

In yet another embodiment, the cell treated in accordance with the inventive method is a cancerous cell. For example, the cell to be treated in accordance with the inventive method can be selected from the group of cancer cells consisting of lung cancer, bronchus cancer, colorectal cancer, prostate cancer, breast cancer, pancreas cancer, stomach cancer, ovarian cancer, urinary bladder cancer, brain or central nervous system cancer, peripheral nervous system cancer, esophageal cancer, cervical cancer, melanoma, uterine or endometrial cancer, cancer of the oral cavity or pharynx, liver cancer, kidney cancer, biliary tract cancer, small bowel or appendix cancer, salivary gland cancer, thyroid cancer, adrenal gland cancer, osteosarcoma, chondrosarcoma, liposarcoma, testes cancer, lymphoma, multiple myeloma, and leukemia. Of course, other types of cancer cells also can be treated in accordance with the inventive method.

When employed in vivo against cancer cells, the invention affords a method of attenuating the progression of a cancer in a patient suffering from cancer by administering to the patient an inhibitor of cardiolipin synthesis under conditions sufficient to inhibit progression of said cancer within said patient. For disseminated or metastasized cancer, the conditions can be satisfied by intravenous administration of the inhibitor of cardiolipin synthesis. For topical cancers, such as melanoma or other skin or epithelial cancers, the method can involve topical application of a composition (.e.g., a gel, magma, creme, suppository, etc.) containing the inhibitor of cardiolipin synthesis. In many applications, the cancer cell or cells to be treated are within or form a tumor or other similar structure.

In such instances in which the cancer cell is within a tumor, the invention affords a method of attenuating the growth of the tumor by exposing the tumor to an inhibitor of cardiolipin synthesis under conditions sufficient to attenuate the growth of said tumor. Ideally, the inventive method is used to treat a cancer manifested as a solid tumor or a tumor associated with soft tissue (i.e., soft tissue sarcoma) in a human. The tumor can be associated with cancers of (i.e., located in) the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily the primary tumor. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non-small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system. The target tissue also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). The tumor can be at any stage, and can be subject to other therapies. The inventive method is useful in treating tumors that have been proven to be resistant to other forms of cancer therapy, such as radiation-resistant tumors. The tumor also can be of any size. A preferred method of treating cancerous tumors in accordance with the inventive method involves direct intratumoral or interstitial injection of a composition containing the inhibitor of cardiolipin synthesis. One known process for achieving such direct injection is via convection enhnanced delivery (see, e.g., U.S. Pat. No. 5,720,720).

Where the method is employed to attenuate the progression of cancer within a patient, or to attenuate the growth of a tumor in a patient, the method need not achieve complete elimination or remission of the cancer or tumor. In this regard, a successful therapeutic treatment can include halting the progression of the cancer or tumor, thereby enlarging the time that the growing cancer or tumor can be treated by other methods. In this regard, the inventive method can be employed adjunctively with other methods and reagents for treating cancerous cells and tumor. For example, the method can be employed in conjunction with radiation therapy of cancers or tumors. Alternatively, the inventive method can be used in conjunction with chemotherapeutic methods. Thus, when used to treat cancer cells, the inventive method can include adjunctively exposing the cell or cells to be treated, or a tumor containing them, with one or more antineoplastic agents or other drugs, many of which are known in the art. For example, drugs or active agents for adjunctive use in conjunction with the inventive method can include anticancer agents (e.g., chemotherapeutic agents), in that they are capable of inducing (either directly or indirectly) cancer cell or tumor cell cytotoxicity. Exemplary anticancer agents include mitoxantrone, taxanes, paclitaxel, camptothecin, camptothecin derivatives (e.g., SN-38), topotecan, gemcitabine, vinorelbine, vinblastine, anthracyclines, adriamycin, capecitabine, doctaxel, didanosine (ddI), stavudine (d4T), antisense oligonucleotides (e.g., c-raf antisense oligonucleotide (RafAON)), antibodies (e.g., herceptin), immunotoxins, hydroxyurea, melphalan, chlormethine, extramustinephosphate, uramustine, ifosfamide, mannomustine, trifosfamide, streptozotocin, mitobronitol, mitoxantrone, methotrexate, 5-fluorouracil, cytarabine, tegafur, idoxide, taxol, daunomycin, daunorubicin, bleomycin, amphotericin, carboplatin, cisplatin, BCNU, vincristine, camptothecin, mitomycin, doxorubicin, etopside, histermine dihydrochloride, tamoxifen, cytoxan, leucovorin, oxaliplatin, irinotecan, raltitrexed, epirubicin, anastrozole, proleukin, sulindac, EKI-569, erthroxylaceae, cerubidine, docetaxel, cytokines (e.g., interleukins), ribozymes, interferons, oligonucleotides, and functional derivatives of the foregoing.

In a preferred embodiment of the invention, an anticancer agent for adjunctive use with the inhibitor of cardiolipin synthesis can be an antisense oligonucleotide, typically comprising at least between about 7 and 13 nucleotides and up to between about 32 and 38 nucleotides (e.g., between about 10 and about 35 nucleotides) directed against a gene encoding a product that promotes tumor initiation and/or progression. A preferred antisense nucleotide targets c-raf. (e.g., a c-raf antisense oligonucleotide (RafAON)). Where such oligonucleotides are included, the formulation can additionally includes at least one drug, such as paclitaxel, mitoxantrone, camptothecins (preferably 7-ethyl-10-hydroxycamptothecin, i.e., SN-3 8) doxorubicin, gemcitabine, vinorelbine, vinblastine, cisplatin, 5-fluorouracil, mitomycin, and adriamycin. Methods of using certain of the aforementioned drugs in formulations to treat cancer are known in the art and are described in, for example, Pathak et al., “Potentiation of the effect of paclitaxel and carboplatin by antioxidant mixture on human lung cancer H520 cells,” J. Am. Coll. Nutr., 21 (5), 416-421 (2002); Socinski et al., “Phase II trial of irinotecan, paclitaxel and carboplatin in patients with previously untreated Stage IIIB/IV nonsmall cell lung carcinoma,” Cancer, 95 (7), 1520-1527 (2002); Lewis et al., “Phase I and pharmacokinetic study of irinotecan in combination with raltitrexed,” Cancer Chemother. Pharmacol., 50 (4), 257-265 (2002); Ricci et al., “Gemcitabine plus epirubicin in patients with advanced urothelial carcinoma who are not eligible for platinum-based regimens,” Cancer, 95 (7), 1444-1450 (2002); Park et al., “Liposome-based drug delivery in breast cancer treatment,” Breast Cancer Res., 4 (3), 95-99 (2002); Thigpen, “The role of gemcitabine-based doublets in the management of ovarian carcinoma,” Semin. Oncol, 29 (1 Suppl. 1), 11-16 (2002); and U.S. Pat. No. 5,744,460, all of which are incorporated herein by reference.

Other drugs or active agents which can be employed adjunctively in the inventive method include agents which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, the alimentary and excretory systems, the histamine system and the central nervous system. Suitable agents may be selected from, for example, proteins, enzymes, hormones, nucleotides, polynucleotides, nucleoproteins, polysaccharides, glycoproteins, lipoproteins, polypeptides, steroids, terpenoids, retinoids, anti-ulcer H2 receptor antagonists, antiulcer drugs, hypocalcemic agents, moisturizers, cosmetics, etc. Active agents can be analgesics; anesthetics; anti-arrythmic agents, antibiotics; antiallergic agents, antifingal agents, antihypertensive agents (e.g., dihydropyridines, antidepressants, cox-2 inhibitors); anticoagulants; antidepressants; antidiabetic agents, anti-epilepsy agents, antiinflammatory corticosteroids; agents for treating Alzheimers or Parkinson's disease; antiulcer agents; anti-protozoal agents, anxiolytics, thyroids, anti-thyroids, antivirals, anoretics, bisphosphonates, cardiac inotropic agents, cardiovascular agents, corticosteroids, diuretics, dopaminergic agents, gastrointestinal agents, hemostatics, hypercholesterol agents, antihypertensive agents; immunosuppressive agents; anti-gout agents, anti-malarials, anti-migraine agents, antimuscarinic agents, antiinflammatory agents, such as agents for treating rheumatology, arthritis, psoriasis, inflammatory bowel disease, Crohn's disease; or agents for treating demyelinating diseases including multiple sclerosis; ophthalmic agents; vaccines (e.g., against influenza virus, pneumonia, hepatitis A, hepatitis B, hepatitis C, cholera toxin B-subunit, typhoid, plasmodium falciparum, diphtheria, tetanus, herpes simplex virus, tuberculosis, HIV, bordetela pertusis, measles, mumps, rubella, bacterial toxoids, vaccinia virus, adenovirus, canary virus, bacillus calmette Guerin, klebsiella pneumonia vaccine, etc.); histamine receptor antagonists, hypnotics, kidney protective agents, lipid regulating agents, muscle relaxants, neuroleptics, neurotropic agents, opioid agonists and antagonists, parasympathomimetics, protease inhibitors, prostaglandins, sedatives, sex hormones (e.g., androgens, estrogens, etc.), stimulants, sympathomimetics, vasodilators, xanthins, and synthetic analogs of these species.

The agents or drugs used adjunctively in connection with the inventive method can be nephrotoxic, such as cyclosporins and amphotericin B, or cardiotoxic, such as amphotericin B and paclitaxel. Additional examples of drugs which may be delivered by way of the inventive composition include, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phemnetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamemasone and its derivatives such as betamethasone, triamcinolone, methyltestosterone, 17-S-estradiol, ethinyl estradiol, ethinyl estradiol 3-methyl ether, prednisolone, 17a-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, niraodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, heparin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, somatotropins (e.g., bovine somatotropin, porcine somatotropin, etc.), oxytocin, vasopressin, GRF, somatostatin, lypressin, pancreozymin, luteinizing hormone, LHRH, LHRH agonists and antagonists, leuprolide, interferons (e.g., o>, |3-, or y-interferon, interferon a-2a, interferon a-2b, and consensus interferon, etc.), interleukins, growth hormones (e.g., human growth hormone and its derivatives such as methione-human growth hormone and des-phenylalanine human growth hormone, bovine growth hormone, porcine growth hormone, insulin-like growth hormone, etc.), fertility inhibitors such as the prostaglandins, fertility promoters, growth factors such as insulin-like growth factor, coagulation factors, pancreas hormone releasing factor, analogs and derivatives of these compounds, and pharmaceutically acceptable salts of these compounds, or their analogs or derivatives.

In the context of the inventive method, a therapeutically effective amount of the inhibitor of cardiolipin synthesis (and any additional adjunctive agent) is administered to a mammalian host, most preferably a human host, to treat a condition, such as cancer, obesity, or cardiovascular disease. A “therapeutically effective amount” means an amount sufficient to show a meaningful benefit in an individual, i.e., promoting at least one aspect of apoptosis, tumor cell cytotoxicity, or treatment, healing, prevention, or amelioration of other relevant medical condition(s) associated with a particular disorder. Therapeutically effective amounts may vary depending upon the biological effect desired in the individual, disorder to be treated, and/or the specific characteristics of the inhibitor of cardiolipin synthesis (and any additional adjunctive agent), and individual. Thus, the attending physician (or other medical professional responsible for administering the composition) will typically decide the amount of inhibitor of cardiolipin synthesis (and any additional adjunctive agent) with which to treat each individual patient.

The inhibitor of cardiolipin synthesis (and any additional adjunctive agent) preferably is included in a pharmaceutical preparation in dosage units. This means that the preparations are in the form of individual parts, for example capsules, pills, suppositories and ampoules, of which the content of the liposome composition corresponds to a fraction or a multiple of an individual dose. The dosage units can contain, for example, 1, 2, 3 or 4 individual doses or a fraction of (e.g., ½, ⅓, or ¼, etc.) of an individual dose. An individual dose preferably contains the amount of the liposome which is given in one administration and which usually corresponds to a whole, a half, a third, or a quarter of a daily dose. In this regard, the liposome should preferably be present in a pharmaceutical preparation at a concentration of about 0.01 to 5 wt. %, about 0.05 to 1 wt. %, about 0.1 to 1.5 wt. %, about 0.2 to 1 wt. %, or about 0.5 to 1 wt. % relative to the total mixture. However, it can be necessary to deviate from the dosages mentioned and in particular to do so as a function of the nature and body weight of the subject to be treated, the nature and the severity of the illness, the nature of the preparation and if the administration of the medicine, and the time or interval over which the administration takes place. Thus it can suffice in some cases to manage with less that the abovementioned amount of active compound, whilst in other cases the abovementioned amount of active compound must be exceeded. The particular required optimum dosage and the type of administration of the inhibitor of cardiolipin synthesis (and any additional adjunctive agent) can be determined by one skilled in the art, by available methods. Suitable amounts are therapeutically effective amounts that do not have excessive toxicity, as determined in empirical studies.

In accordance with the inventive method, the inhibitor of cardiolipin synthesis (and any additional adjunctive agent) desirably is formulated into a pharmaceutical composition comprising a physiologically acceptable (e.g., a pharmaceutically or pharmacologically acceptable) carrier (e.g., excipient or diluent). Any suitable physiologically acceptable carrier can be used within the context of the invention, and such carriers are well known in the art. Most preferably, the inventive method employs a non-toxic, inert physiologically-acceptable carrier. Such carriers are known in the art and include, for example, semi-solid or liquid diluents, fillers and formulation auxiliaries of all kinds. The carrier typically will be liquid, but also can be solid, or a combination of liquid and solid components. The choice of carrier will be determined, at least in part, by the location of the target tissue and/or cells, and the particular method used to administer the composition.

Typically, such compositions can be prepared as injectables, either as liquid solutions or suspensions. Solid forms suitable for using to prepare solutions or suspensions upon the addition of a liquid prior to injection can also be prepared, and the preparations can also be emulsified. The pharmaceutical forms suitable for injectable use include sterile aqueous solutions or dispersions, formulations including sesame oil, peanut oil or aqueous propylene glycol, and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of cardiolipin synthesis (and other adjunctive agents) for use in the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like. The composition can further comprise any other suitable components, especially for enhancing the stability of the composition and/or its end-use. Accordingly, there is a wide variety of suitable formulations of the composition of the invention. The following formulations and methods are merely exemplary and are in no way limiting. Formulations in accordance with these exemplary types can be manufactured in the usual manner according to known methods, for example by mixing the inhibitor of cardiolipin synthesis (and any other adjunctive active agents) with the appropriate excipient or excipients.

For oral administration, the inhibitor of cardiolipin synthesis (and other adjunctive agents) can be formulated as tablets, capsules, lozenges, powders, syrups, aqueous solutions, suspensions, and the like. Carriers such as lactose, sodium citrate, and salts of phosphoric acid can be used to prepare tablets. Further, disintegrants such as starch, and lubricating agents, such as magnesium stearate, sodium lauryl sulfate and talc can be included. Diluents such as lactose and high molecular weight polyethylene glycols can be used in the preparation of dosages in capsule form. The active ingredient can be combined with emulsifying and suspending agents to generate aqueous suspensions for oral use. Flavoring agents such as sweeteners can be added, as desired.

For topical (i.e., dermal) administration, the inhibitor of cardiolipin synthesis (and other adjunctive agents) can be provided in the form of gels, oils, and emulsions by the addition of suitable water-soluble or water-insoluble excipients, for example polyethylene glycols, certain fats, and esters or mixtures of these substances. Suitable excipients are those in which the liposome composition is sufficiently stable to allow for therapeutic use.

Formulations suitable for anal administration can be prepared as suppositories by mixing the inhibitor of cardiolipin synthesis (and other adjunctive agents) with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

Formulations suitable for administration of the inhibitor of cardiolipin synthesis (and other adjunctive agents) via inhalation include aerosol formulations. The aerosol formulations can be placed into pressurized acceptable propellants, such as dichlorodifluoromethane, propane, nitrogen, and the like. They also can be formulated as non-pressurized preparations, for delivery from a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

In addition to the inhibitor of cardiolipin synthesis (and other adjunctive agents), the composition can comprise additional therapeutic or biologically-active agents. For example, therapeutic factors (e.g., antibodies) useful in the treatment of a particular indication can be present. Factors that control inflammation, such as ibuprofen or steroids, can be part of the composition to reduce swelling and inflammation associated with in vivo administration of the inhibitor of cardiolipin synthesis (and other adjunctive agents) and physiological distress. Immune system suppressors can be administered with the composition to reduce any immune response to the antibody itself or associated with a disorder. Alternatively, immune enhancers can be included in the composition to up-regulate the body's natural defenses against disease. Moreover, cytokines can be administered with the composition to attract immune effector cells to a disease (e.g., tumor) site.

Preferred formulations for use in vivo can include liposomes. Accordingly, for use in the inventive method, the invention also provides a pharmaceutical composition including an inhibitor of cardiolipin synthesis (e.g., an antibody, genetic vector, polynucleotide, or small molecule inhibitor of cardiolipin synthesis, such as described above) and a liposome. Desirably, the inhibitor of cardiolipin synthesis is entrapped in the liposome, such as within the lipid fraction or the lumen of the liposomes within the composition.

Where such liposomal formulations of an antibody, genetic vector, polynucleotide, or small molecule inhibitor of cardiolipin synthesis are employed, it is desirable for the liposomal fraction to contain cardiolipin among the lipids. The cardiolipin can be a natural or a synthetic cardiolipin and it can be neutral, or charged positively or negatively, as desired. The precise formulation of the inhibitor of cardiolipin synthesis, however, is not critical to the inventive method, and it is within the ordinary skill of the art to formulate active agents, such as antibodies, genetic vectors, antisense polynucleotides, and small molecule “drugs” into such formulations for intravenous injection, or for other modes of application, into liposomal formulations.

In a preferred embodiment of the invention, the liposome composition is formulated for injection. In this regard, the formulation desirably is suitable for intratumoral administration, but also can be formulated for intravenous injection, intraperitoneal injection, subcutaneous injection, and the like. In this manner, for example, liposome formulations containing two or more anticancer drugs may be injected directly into tumor tissue for delivery of the anticancer drugs directly to cancer cells. In some cases, particularly after resection of a tumor, the liposome formulation can be implanted directly into the resulting cavity or may be applied to the remaining tissue as a coating. In cases in which the liposome formulation is administered after surgery, it is possible to utilize liposomes having larger diameters of about 1 micron since they do not have to pass through the vasculature.

All references, including publications, patent applications, and patents, cited herein, including those cited above in the text of the specification, and in the following list, are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of attenuating the progression of a cancer in a patient suffering from cancer, said method comprising administering to said patient an inhibitor of cardiolipin synthesis under conditions sufficient to inhibit progression of said cancer within said cancer.

2. The method of claim 1, wherein the cancer comprises a tumor and wherein said inhibitor of cardiolipin synthesis is administered to said patient by direct injection at the site of the tumor.

3. The method of claim 2, wherein said injection is interstitial.

4. A method of attenuating the growth of a tumor, said method comprising administering an inhibitor of cardiolipin synthesis to said tumor under conditions sufficient to attenuate the growth of said tumor.

5. The method of claim 4, wherein the growth of the tumor is caused by cancer.

6. The method of claim 5, wherein the tumor is in vivo.

7. The method of any of claims 1-6 wherein the inhibitor of cardiolipin synthesis is administered within a pharmaceutical composition comprising said inhibitor of cardiolipin synthesis and a pharmaceutically acceptable carrier.

8. The method of claim 7 wherein the inhibitor of cardiolipin synthesis is selected from the group of compounds consisting of 1-Decanoyl-sn-glycero-3-phosphorylcholine, 1-O-octadecyl-2-O-methyl-rac-glycero-3-phosphocholine, hexadecylphosphocholine, Lysophosphatidic acid, palmitate, N-(4-hydroxyphenyl)retinamide, Phosphatidyl-3,4-Dihydroxybutyl-1-phosphate, Phosphatidylserine, Sphingosine-1-phosphate, and Sulfoquinovosyldiacylglycerol.

9. The method of claim 7 wherein the inhibitor of cardiolipin synthesis is an antibody.

10. The method of claim 7 wherein the inhibitor of cardiolipin synthesis is a polynucleotide having a sequence antisense to the coding sequence of an enzyme in the cardiolipin synthesis pathway.

11. The method of claim 10 wherein the enzyme is selected from the group of enzymes consisting of phosphatidylglycerophosphate synthase, phosphatidylglycerophosphate phosphatase and cardiolipin synthase.

12. The method of claim 7 wherein the inhibitor of cardiolipin synthesis is a polynucleotide having a sequence antisense to the regulatory sequence of an enzyme in the cardiolipin synthesis pathway.

13. The method of claim 12 wherein the enzyme is selected from the group of enzymes consisting of phosphatidylglycerophosphate synthase, phosphatidylglycerophosphate phosphatase and cardiolipin synthase.

14. The method of claim 1, wherein the cancer is selected from a group consisting of lung cancer, bronchus cancer, colorectal cancer, prostate cancer, breast cancer, pancreas cancer, stomach cancer, ovarian cancer, urinary bladder cancer, brain or central nervous system cancer, peripheral nervous system cancer, esophageal cancer, cervical cancer, melanoma, uterine or endometrial cancer, cancer of the oral cavity or pharynx, liver cancer, kidney cancer, biliary tract cancer, small bowel or appendix cancer, salivary gland cancer, thyroid cancer, adrenal gland cancer, osteosarcoma, chondrosarcoma, liposarcoma, testes cancer, lymphoma, multiple myeloma and leukemia.

15. The method of any of claim 1, further comprising administering an anti-neoplastic agent.

16. The method of claim 15, wherein the anti-neoplastic agent is selected from the group of anti-neoplastic agents consisting of mitoxantrone, taxanes, paclitaxel, camptothecin, camptothecin derivatives, topotecan, gemcitabine, vinorelbine, vinblastine, anthracyclines, adriamycin, capecitabine, doctaxol, didanosine (ddl), stavudine (d47), antisense oligonucleotides, antibodies, immunotoxins, hydroxyurea, melphalan, chlormethine, extramustinephosphate, uramustine, ifosfamide, mannomustine, trifosfamide, streptozotocin, mitobronitol, mitoxantrone, methotrexate, 5-fluorouracil, cytarabine, tegafur, idoxide, taxol, daunomycin, daunorubicin, bleomycin, amphotericin, carboplatin, cisplatin, BCNU, vincristine, mitomycin, doxorubicin, etopside, histermine dihydrochloride, tamoxifen, cytoxan, leucovorin, oxaliplatin, irinotecan, raltitrexed, epirubicin, anastrozole, proleukin, sulindac, EKI-569, erthroxylaceae, cerubidine, docetaxel, cytokines, ribozymes, interferons, oligonucleotides, and functional derivatives and combinations thereof.

17. A method of attenuating the progression of obesity in a patient suffering from obesity, said method comprising administering to said patient an inhibitor of cardiolipin synthesis under conditions sufficient to inhibit proliferation or growth of adipose cells within said patient.

18. The method of claim 17, wherein the adipose cells comprise adipose tissue and wherein said inhibitor of cardiolipin synthesis is administered to the patient by direct injection into the adipose tissue.

19. A method of attenuating the progression of an adipose growth, said method comprising administering an inhibitor of cardiolipin synthesis to the adipose growth under conditions sufficient to attenuate the progression of said adipose growth.

20. The method of claim 19, wherein the adipose growth is in vivo.

21. A method of treating a patient suffering from a cardiovascular disease characterized by the buildup of fatty plaque deposits in vascular walls, said method comprising administering to said patient an inhibitor of cardiolipin synthesis under conditions sufficient to inhibit proliferation of fatty plaque deposits in vascular walls within said patient.

22. A method of treating a patient suffering from a cardiovascular disease characterized by the buildup of fatty plaque deposits in vascular walls, said method comprising administering to said patient an inhibitor of cardiolipin synthesis under conditions sufficient to reduce the amount of plaque present within the vascular tissue.

23. The method of any of claims 17-22 wherein the inhibitor of cardiolipin synthesis is administered within a pharmaceutical composition comprising said inhibitor of cardiolipin synthesis and a pharmaceutically acceptable carrier.

24. The method of claim 23 wherein the inhibitor of cardiolipin synthesis is selected from the group of compounds consisting of 1-Decanoyl-sn-glycero-3-phosphorylcholine, 1-O-octadecyl-2-O-methyl-rac-glycero-3-phosphocholine, hexadecylphosphocholine, Lysophosphatidic acid, palmitate, N-(4hydroxyphenyl)retinamide, Phosphatidyl-3,4-Dihydroxybutyl-1-phosphate, Phosphatidylserine, Sphingosine-1-phosphate, and Sulfoquinovosyldiacylglycerol.

25. The method of claim 23 wherein the inhibitor of cardiolipin synthesis is an antibody.

26. The method of claim 23 wherein the inhibitor of cardiolipin synthesis is a polynucleotide having a sequence antisense to the coding sequence of an enzyme in the cardiolipin synthesis pathway.

27. The method of claim 26 wherein the enzyme is selected from the group of enzymes consisting of phosphatidylglycerophosphate synthase, phosphatidylglycerophosphate phosphatase and cardiolipin synthase.

28. The method of claim 23 wherein the inhibitor of cardiolipin synthesis is a polynucleotide having a sequence antisense to the regulatory sequence of an enzyme in the cardiolipin synthesis pathway.

29. The method of claim 28 wherein the enzyme is selected from the group of enzymes consisting of phosphatidylglycerophosphate synthase, phosphatidylglycerophosphate phosphatase and cardiolipin synthase.

30. A pharmaceutical composition, comprising an inhibitor of cardiolipin synthesis and a liposomal carrier.

31. The composition of claim 30, wherein the inhibitor of cardiolipin synthesis is selected from the group of compounds consisting of 1-Decanoyl-sn-glycero-3-phosphorylcholine, 1-O-octadecyl-2-O-methyl-rac-glycero-3-phosphocholine, hexadecylphosphocholine, Lysophosphatidic acid, palmitate, N-(4-hydroxyphenyl)retinamide, Phosphatidyl-3,4-Dihydroxybutyl-1-phosphate, Phosphatidylserine, Sphingosine-1-phosphate, and Sulfoquinovosyldiacylglycerol.

32. The composition of claim 30, wherein the inhibitor of cardiolipin synthesis is an antibody.

33. The composition of claim 30, wherein the inhibitor of cardiolipin synthesis is a polynucleotide having a sequence antisense to the coding sequence of an enzyme in the cardiolipin synthesis pathway.

34. The composition of claim 33, wherein the enzyme is selected from the group of enzymes consisting of phosphatidylglycerophosphate synthase, phosphatidylglycerophosphate phosphatase and cardiolipin synthase.

35. The composition of claim 30, wherein the inhibitor of cardiolipin synthesis is a polynucleotide having a sequence antisense to the regulatory sequence of an enzyme in the cardiolipin synthesis pathway.

36. The composition of claim 35 wherein the enzyme is selected from the group of enzymes consisting of phosphatidylglycerophosphate synthase, phosphatidylglycerophosphate phosphatase and cardiolipin synthase.

37. The composition of claim 30 further comprising an anti-neoplastic agent.

38. The composition of claim 37, wherein the anti-neoplastic agent is selected from the group of anti-neoplastic agents consisting of mitoxantrone, taxanes, paclitaxel, camptothecin, camptothecin derivatives (e.g., SN-38), topotecan, gemcitabine, vinorelbine, vinblastine, anthracyclines, adriamycin, capecitabine, docetaxel, didanosine (ddI), stavudine (d47), antisense oligonucleotides (e.g., c-raf antisense oligonucleotide (RafAON)), antibodies (e.g., herceptin), immunotoxins, hydroxyurea, melphalan, chlormethine, extramustinephosphate, uramustine, ifosfamide, mannomustine, trifosfamide, streptozotocin, mitobronitol, mitoxantrone, methotrexate, 5-fluorouracil, cytarabine, tegafur, idoxide, taxol, daunomycin, daunorubicin, bleomycin, amphotericin, carboplatin, cisplatin, BCNU, vincristine, mitomycin, doxorubicin, etopside, histermine dihydrochloride, tamoxifen, cytoxan, leucovorin, oxaliplatin, irinotecan, raltitrexed, epirubicin, anastrozole, proleukin, sulindac, EKI-569, erthroxylaceae, cerubidine, docetaxel, cytokines (e.g., interleukins), ribozymes, interferons, oligonucleotides, and functional derivatives and combinations thereof.
